(19) United States
(12) Patent Application Publication
(10) Pub. No.: US 2024/0353590 A1
(43) Pub. Date: Oct. 24, 2024

(54) CALIBRATION METHOD AND SYSTEM FOR LONG-WAVE RADIOMETER

(71) Applicant: Institute of Atmospheric Physics, Chinese Academy of Scicences., Beijing (CN)

(72) Inventor: Jianhui Bai, Beijing (CN)

(21) Appl. No.: 18/534,577

(22) Filed: Dec. 9, 2023

(30) Foreign Application Priority Data
Apr. 24, 2023 (CN) 2023104455109

(51) Int. Cl. G01W 1/18; G01N 33/00

(52) U.S. Cl. CPC G01W 1/18; G01N 33/004

(57) ABSTRACT

A calibration method and a system for long-wave radiometers are provided, which relate to the field of calibration technologies for long-wave radiometers, and the method includes: obtaining, by using long-wave radiation values at a top of atmosphere (TOA) as a standard, data of meteorological parameters and carbon dioxide (CO₂) flux measured at a target station; determining a target energy action state for expressing physical, chemical and biological processes involved in radiation transmission and interaction between the data and radiation based on change rules of radiation, the meteorological parameters and the CO₂ flux and interrelation of them; obtaining a calibration coefficient based on the target energy action state and values and value ranges of temperature and humidity, ground water vapor pressure and the CO₂ flux to calibrate the long-wave radiometer; the calibration work can be performed at the station during routine measurement, and a continuity of the measurement can be ensured.

### TECHNICAL FIELD

The disclosure relates to the field of calibration technologies for long-wave radiometers, and more particularly to a calibration method and a system for long-wave radiometers.

### BACKGROUND

At present, for a calibration work of a long-wave radiometer all over the world, it needs to send a long-wave radiometer used in a station to a national designated department, the long-wave radiometer used in the station and a standard long-wave radiometer meeting international and domestic long-wave radiation standards are used to measure long-wave radiation under clear and cloudless conditions to obtain measured results, the measured results of the two long-wave radiometers are compared to obtain a comparative result, a new calibration value of the long-wave radiometer to-be-calibrated (i.e., the long-wave radiometer used in the station) is determined according to the comparative result to complete the calibration work of the long-wave radiometer, and the calibrated long-wave radiometer is transported back to the station for future measurement. This calibration method needs to rely on calibration resources (e.g., the standard long-wave radiometer, a calibration device and a calibration software), and a large amount manpower and transportation, the daily maintenance and preservation conditions of the standard long-wave radiometer are harsh, the station needs to equip another long-wave radiometer for developing a normal measurement work during the calibration work, and the measurement work is interrupted when a meter is replaced. Therefore, it is urgent to design a new calibration method of the long-wave radiometer for solving reliance of the standard long-wave radiometer during the calibration work of international and domestic long-wave radiometers, and conserving manpower, material resources and transportation costs used in previous calibration works.

### SUMMARY

In order to solve problems in the related art, a purpose of the disclosure is to provide a calibration method and a system for long-wave radiometers, a long-wave radiation calculation model and a calibration method of a long-wave radiometer are determined based on station measurement data (i.e., meteorological parameters and carbon dioxide (CO₂) flux) and an energy balance principle (i.e., radiation energy balance principle), so as to solve reliance of a standard long-wave radiometer in the related art.

In order to achieve the above technical purpose, the disclosure provides a calibration method for a long-wave radiometer, and the method includes:
a collection process, including: obtaining, by using a long-wave radiation value at a top of atmosphere (TOA) as a standard, data of the meteorological parameters and the CO₂ flux measured at a target station;
a screening process, including: determining, based on change rules of radiation, the meteorological parameters and the CO₂ flux, a target energy action state for expressing physical, chemical and biological processes involved in a radiation transmission and an interaction between the data and the radiation by obtaining an interrelation among the radiation, the meteorological parameters and the CO₂ flux; and
a calibration process, including: obtaining, based on the target energy action state, a calibration coefficient by determining values and value ranges of temperature and humidity, a ground water vapor pressure and the CO₂ flux, to calibrate the long-wave radiometer.

In an embodiment, the collection process specifically includes: obtaining the data of the meteorological parameters and the CO₂ flux measured at night at the target station.

In an embodiment, the screening process further includes: selecting data within 2 times standard deviation for the CO₂ flux; obtaining, based on regenerated data, a standard deviation after each cycle calculation; and adopting a data process method synchronized with the CO₂ flux for the meteorological parameters.

In an embodiment, the calibration process specifically includes: obtaining, based on the temperature and humidity, the ground water vapor pressure and the CO₂ flux, an absorption term and a CO₂ term for representing attenuation involved in the radiation transmission, and obtaining, based on a radiation energy balance principle, the calibration coefficient to calibrate the long-wave radiometer.

In an embodiment, in a process of obtaining the absorption term, a formula of the absorption term e^{-kWm} is expressed as follows:

$${e}^{- k W m} = 1 - Δ {SI}_{0} cos ( Z ) ;$$

where I₀ represents a solar constant, Z represents a solar zenith angle, ΔS represents a solar radiation flux density of total absorption in atmospheric column, and ΔS=0.172 (mW×0.1×30)^{0.303}, k represents a water vapor absorption coefficient, m represents an air mass, and W represents a water vapor content in the atmospheric column.

In an embodiment, in a process of obtaining the CO₂ term, the CO₂ term is expressed as e^{-0.1bRetm}; where b represents an attenuation coefficient, and Re represents the CO₂ flux; and t represents a sampling time.

In an embodiment, a process of obtaining the calibration coefficient includes: obtaining, based on the radiation energy balance principle, the calibration coefficient by obtaining an energy equation between the CO₂ term and the absorption term in a horizontal plane above a canopy layer, where a formula of the energy equation is expressed as follows:

$${e}^{- 0 \text{.1} bRetm} \times cos ( Z ) = {C}_{1} {e}^{- k w m} \times cos ( Z ) + {C}_{0} ;$$

where C₁ and C₀ represent parameters related to various factors at the TOA.

In an embodiment, in the process of obtaining the calibration coefficient, the calibration coefficient is expressed as follows:

$$\begin{matrix}L {R}_{0} / LR ; \\ LR = {C}_{1} + \vert {C}_{0} \vert ;\end{matrix}$$

where LR represents the long wave radiation value, and LR₀ represents long-wave radiation emitted by the canopy layer.

The process of obtaining the calibration coefficient further includes: obtaining, based on a Boltzmann equation, the long-wave radiation emitted by the canopy layer by measuring temperature and emissivity at the canopy layer.

In an embodiment, in the process of the calibration coefficient, when C₁ and C₀ are positive and negative values, respectively, the calibration coefficient is determined based on the energy equation.

The disclosure further provides a calibration system for a long-wave radiometer, and the system includes a collection module, a screening module and a calibration module.

The collection module is configured to obtain, by using a long-wave radiation value at the TOA as a standard, data of meteorological parameters and CO₂ flux measured at a target station.

The screening module is configured to determine, based on change rules of radiation, the meteorological parameters and the CO₂ flux, a target energy action state for expressing physical, chemical and biological processes involved in a radiation transmission and an interaction between the data and the radiation by obtaining an interrelation among the radiation, the meteorological parameters and the CO₂ flux.

The calibration module is configured to obtain, based on the target energy action state, a calibration coefficient by determining values and value ranges of temperature and humidity, a ground water vapor pressure and the CO₂ flux, to calibrate the long-wave radiometer.

In an embodiment, each of the collection module, the screening module and the calibration module is embodied by software stored in at least one memory and executable by at least one processor.

The disclosure provides the following beneficial effects.

A calibration work can be developed on-site at the target station during routine measurement, which ensures a continuity of a measurement work; calibration devices, supporting equipment (software and hardware) thereof, and backup radiometers for station working are conserved, and manpower, material resources and transportation costs of the radiometers are also conserved.

The standard of long-wave radiation used in the calibration work of the disclosure is the long-wave radiation value at the TOA, the long-wave radiation value at the TOA can be obtained by the Boltzmann equation and temperature of ground (i.e., canopy layer), the value is reliable, required temperature data is easy to timely obtain from the daily measurements of the station, thus solving practical problems of relying on standard long-wave radiometers for the calibration work or going to designated departments for the calibration work, and improving a work efficiency of the calibration work.

### BRIEF DESCRIPTION OF DRAWINGS

In order to clearly describe technical solutions in embodiments of the disclosure or related art, drawings required in the embodiment descriptions will be introduced below. Obviously, the drawings described below are merely some embodiments of the disclosure, for those skilled in the art, other drawings can be obtained according to the drawings without creative work.

FIG. 1 illustrates a flowchart of a specific implementation of a calibration method for long-wave radiometer according to an embodiment of the disclosure.

FIG. 2 illustrates a flowchart of a calibration method for long-wave radiometer according to an embodiment of the disclosure.

### DETAILED DESCRIPTION OF EMBODIMENTS

In order to make purposes, technical solutions and advantages in embodiments of the disclosure clearer, the technical solutions in the embodiments of the disclosure will be clearly and completely described in conjunction with drawings in the embodiments of the disclosure below. Obviously, the described embodiments are merely some embodiments of the disclosure, not all of them. Components in the embodiments of the disclosure described and shown in the drawings can be arranged and designed as different configurations. Therefore, the following detail descriptions for the embodiments of the disclosure provided in the drawings are not aimed to limit a scope of protection of the disclosure, but only represent selected embodiments of the disclosure. Based on the embodiments of the disclosure, all other embodiments obtained by those skilled in the art without creative work fall within the scope of protection of the disclosure.

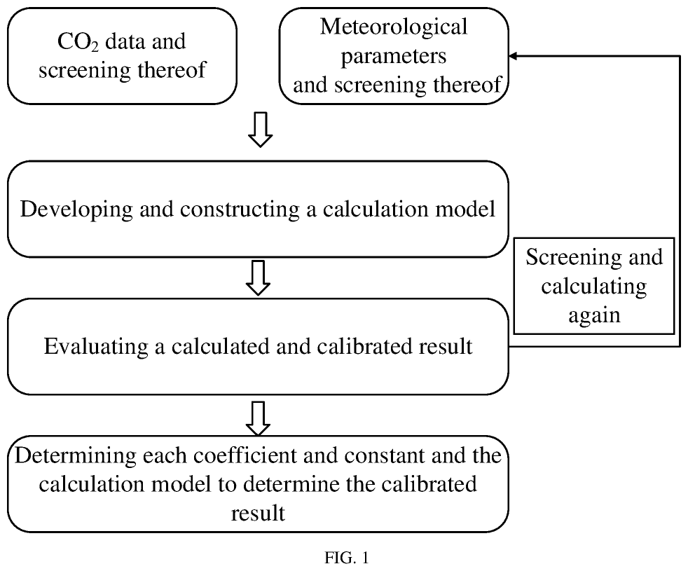

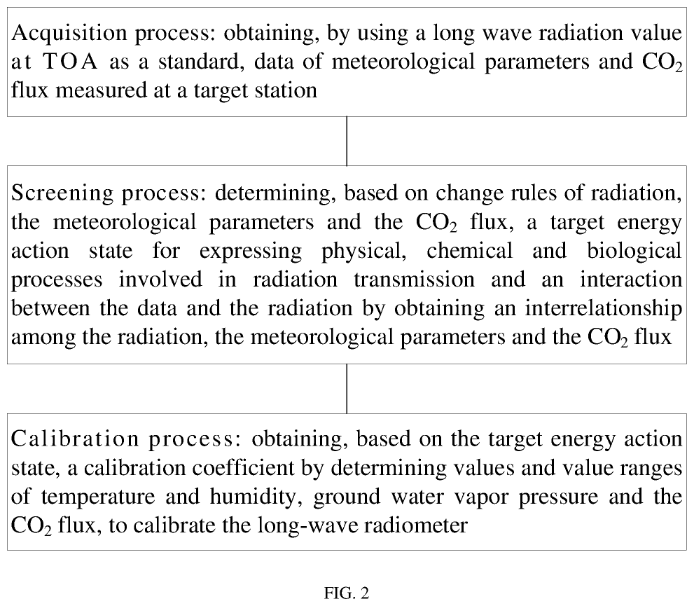

As shown in FIGS. 1-2, the disclosure provides a calibration method for a long-wave radiometer, and the method includes: a collection process, a screening process and a calibration process.

In the collection process, data of meteorological parameters and carbon dioxide (CO₂) flux measured at a target station is obtained, and a long-wave radiation value at a top of atmosphere (TOA) is used as a standard.

In the screening process, a target energy action state is determined based on change rules of radiation, the meteorological parameters and the CO₂ flux by obtaining an interrelation among the radiation, the meteorological parameters and the CO₂ flux, and the target energy action state is used to express physical, chemical and biological processes involved in a radiation transmission and an interaction between the data and the radiation.

In the calibration process, a calibration coefficient is obtained based on the target energy action state by determining values and value ranges of temperature and humidity, a ground water vapor pressure and the CO₂ flux, and the calibration coefficient is used to calibrate the long-wave radiometer.

In an embodiment, during the collection process of the disclosure, the data of the meteorological parameters and the CO₂ flux measured at night at the target station is obtained.

In an embodiment, during the screening process of the disclosure, data within 2 times standard deviation is selected for the CO₂ flux; and a new standard deviation is obtained based on regenerated data after each cycle calculation.

A data process method synchronized with the CO₂ flux is adopted for the meteorological parameters.

In an embodiment, during the calibration process of the disclosure, an absorption term and a CO₂ term for representing attenuation involved in the radiation transmission are obtained based on the temperature and humidity, the ground water vapor pressure and the CO₂ flux, and the calibration coefficient is obtained based on a radiation energy balance principle to calibrate the long-wave radiometer.

In an embodiment, in a process of obtaining the absorption term, a formula of the absorption term e^{-kWm} is expressed as follows:

$${e}^{- k W m} = 1 - Δ {SI}_{0} cos ( Z ) ;$$

where I₀ represents a solar constant, Z represents a solar zenith angle, ΔS represents a solar radiation flux density of total absorption in atmospheric column, and ΔS=0.172 (mW×0.1×30)^{0.303}, k represents a water vapor absorption coefficient, m represents an air mass, and W represents a water vapor content in the whole atmospheric column.

In an embodiment, in a process of obtaining the CO₂ term, the CO₂ term is expresses as follows:
e^{-0.1bRetm}.
where b represents an attenuation coefficient, and Re represents the CO₂ flux; and t represents a sampling time.

In an embodiment, in a process of obtaining the calibration coefficient of the disclosure, the calibration coefficient is obtained based on the radiation energy balance principle by obtaining an energy equation between the CO₂ term and the absorption term in a horizontal plane above a canopy layer; specifically, the energy equation is expressed as follows:

$${e}^{- 0 \text{.1} bRetm} \times cos ( Z ) = {C}_{1} {e}^{- k w m} \times cos ( Z ) + {C}_{0} ;$$

where C₁ and C₀ represent parameters (energy) related to various factors at the TOA.

In an embodiment, in the process of obtaining the calibration coefficient of the disclosure, the calibration coefficient is expressed as follows:

$$\begin{matrix}L {R}_{0} / LR ; \\ LR = {C}_{1} + \vert {C}_{0} \vert ;\end{matrix}$$

where LR represents the long wave radiation value, and LR₀ represents long-wave radiation emitted by the canopy layer.

The long-wave radiation emitted by the canopy layer is obtained based on a Boltzmann equation by measuring temperature and emissivity at the canopy layer.

In an embodiment, in the process of obtaining the calibration coefficient of the disclosure, the calibration coefficient is determined by the energy equation when C₁ and C₀ are positive and negative values, respectively.

The disclosure provides a calibration system for a long-wave radiometer, and the system includes a collection module, a screening module and a calibration module.

The collection module is configured to obtain data of meteorological parameters and CO₂ flux measured at a target station by using a long-wave radiation value at the TOA as a standard.

The screening module is configured to determine a target energy action state based on change rules of radiation, the meteorological parameters and the CO₂ flux by obtaining an interrelation among the radiation, the meteorological parameters and the CO₂ flux, and the target energy action state is used to express physical, chemical and biological processes involved in a radiation transmission and an interaction between the data and the radiation.

The calibration module is configured to obtain a calibration coefficient based on the target energy action state by determining values and value ranges of temperature and humidity, a ground water vapor pressure and the CO₂ flux, to thereby calibrate the long-wave radiometer.

In an embodiment 1, the new calibration method for the long-wave radiometer uses the long-wave radiation value at the TOA as the standard, and can be developed at a local station, the method uses the data of the meteorological parameters and the CO₂ flux measured at night at the station, the target energy action state for expressing the physical, chemical and biological processes involved in the radiation transmission and the interaction between the data and the radiation is continuously searched and determined based on the change rules of the radiation, the meteorological parameters and the CO₂ flux, and principles such as unification and harmony of the radiation, the meteorological parameters and the CO₂ flux, thus determining the values and value ranges of the meteorological parameters (e.g., the temperature and humidity and the ground water vapor pressure) and the CO₂ flux, and ultimately determining each coefficient and the calibration coefficient of a calculation model.

In a process of screening the above data, it is necessary to perform multiple cycles one by one and in sequence. The process includes the following steps 1-5. In step 1, the data within 2 times standard deviation is selected for the CO₂ flux; a new standard deviation is obtained based on the regenerated data after each cycle calculation. In step 2, the data synchronized with the CO₂ flux is adopted for the meteorological parameters. In step 3, the absorption term and the CO₂ term involved attenuation in the radiation transmission process are calculated for the screened data such as the ground water vapor pressure and the CO₂ flux, and the calculation model is determined based on the radiation energy balance principle, that is, coefficients, constants and the calibration coefficient of the calculation model are determined. Each calculation is as follows.

The formula for calculating the absorption term e^{-kWm} is expressed as: e^{-kWm}=1−ΔSI₀ cos(Z); I₀ represents the solar constant, and I₀=1367 watts per square meter (W·m⁻²); Z represents the solar zenith angle; ΔS represents the solar radiation flux density of total absorption in the atmospheric column, and ΔS=0.172 (mW×0.1×30)^{0.303} calories per square centimeter per minute (cal·cm⁻²·min⁻¹, and 1 cal·cm⁻²·min⁻¹=696.7 W·m⁻²); k represents the water vapor absorption coefficient (m⁻¹); m represents the air mass; W represents the total water vapor content in the atmospheric column (W=0.21E); E represents the ground water vapor pressure (hectopascal abbreviated as hPa); and cos(Z) represents a conversion of the absorption term and the CO₂ term to the horizontal plane. e is an e index.

A formula of the CO₂ term is expresses as: e^{-0.1bRetm}; b represents the attenuation coefficient (b=1), and Re represents the CO₂ flux (milligram CO₂ per square meter per second abbreviated as mg CO₂m⁻²s⁻¹); and t represents the sampling time (1 hour).

The energy equation (i.e., calculation model) between the CO₂ term and the absorption term in the horizontal plane above the canopy layer is obtained based on the radiation energy balance principle, and the energy equation is expresses as follows:

$${e}^{- 0 \text{.1} bRetm} \times cos ( Z ) = {C}_{1} {e}^{- k w m} \times cos ( Z ) + {C}_{0} .$$

Relevant parameters of the ground and TOA, and the CO₂ flux are calculated by using the calculation model and the screened data, and the relevant parameters include: C₁, C₀ and the long-wave radiation value LR=C₁+|C₀|; the long-wave radiation (LR₀) emitted by the ground (i.e., canopy layer) is calculated by using the Boltzmann equation (i.e., εσT⁴, where E represents the emissivity of the ground (i.e., canopy layer), a represents a Stefan-Boltzmann constant (5.67×10⁻⁸ watts per meter² per kelvin⁴ abbreviated as W·m⁻²·K⁻⁴), T represents the temperature of the ground (i.e., canopy layer)) and synchronous temperature and emissivity data of the canopy layer measured at the station. The calculated CO₂ flux is compared to a measurement value, a calculation effect and a calibration quality of the calculation model are evaluated, involved parameters include a calculation value, the measurement value and a deviation between the calculation value and the measurement value (e.g., an average value, a median, an absolute deviation and a range thereof, a relative deviation and a range thereof, a root mean square error and a standard deviation). The calibration coefficient LR₀/LR is obtained.

In step 4, processes of steps 1-3 are repeated when expected effects of a calculation deviation and the calibration coefficient are not achieved (i.e., the relative deviation is smaller than 15 percent (%), and the calibration coefficient tends to stabilize). Various data is further screened and eliminated according to data after each cycle. A new standard is adopted based on the new data (e.g., the average value, the median, the range and the standard deviation) for a new round for screening and eliminating data. The calculation value of the CO₂ flux is compared to the measurement value, and the calculation effect and the calibration quality of the calculation model are evaluated again.

In step 5, after multiple cycles and calculations, the cycle is stopped until reasonable and minimum calculation deviation (e.g., the absolute deviation, the relative deviation, the root mean square and the standard deviation), relatively stable coefficients and C₁+|C₀| are obtained. The coefficient C₁ represents the long-wave radiation at the TOA related to substances in the atmosphere, and C₀ represents the long-wave radiation emitted by the ground (i.e., canopy layer) to the TOA.

A condition that the coefficients determined in the calculation method ultimately need to be satisfied is that C₁ and C₀ are positive and negative values, respectively.

The calibration coefficient of the long-wave radiometer is LR₀/LR.

The new calibration work can be performed at the station (i.e., continuous daily observation work), and efficiencies of the calibration work and the observation work can be improved (e.g., a long-wave radiometer is conserved, steps and operational procedures for transfer of solar long-wave radiation standards from international to domestic sources are conserved). A calibration value and calibration quality of the new calibration method can be determined and tested by a calculation value of the long-wave radiation at the TOA, and the calculation value can be obtained by using the Boltzmann equation and surface temperature; and reliance for the standard long-wave radiometer in the calibration work of the long-wave radiometer of international and domestic is solved, and manpower, material resources, and transportation costs used in the previous calibration work are also conserved.

The disclosure is described according to a flowchart and/or a block diagram of the method, the device (i.e., system), and a computer program product of the embodiment of the disclosure. It should be understood that each flow path in the flowchart and/or each block in the block diagram, and a combination of flow paths in the flowchart and/or blocks in the block diagram can be achieved by computer program instructions. The computer program instructions can be provided to a general computer, a special computer, an embedded processor or a processor of another programmable data processing device to generate a machine, and a device for achieving a specified function in one or more flow paths of the flowchart and/or one or more blocks of the block diagram is generated by instructions executed by the general computer or the processor of another programmable data processing device.

Obviously, various modifications and variants can be performed on the disclosure by those skilled in the art without depart from spirit and scope of the disclosure. In this way, if these modifications and variants of the disclosure fall within scope of the claims and their equivalent technologies, the disclosure is also intended to include these modifications and variants.

What is claimed is:

1. A calibration method for a long-wave radiometer, comprising:
a collection process, comprising: obtaining, by using a long wave radiation value at a top of atmosphere (TOA) as a standard, data of meteorological parameters and carbon dioxide (CO₂) flux measured at a target station;
a screening process, comprising: determining, based on change rules of radiation, the meteorological parameters and the CO₂ flux, a target energy action state for expressing physical, chemical and biological processes involved in a radiation transmission and an interaction between the data and the radiation, by obtaining an interrelationship among the radiation, the meteorological parameters and the CO₂ flux; and
a calibration process, comprising: obtaining, based on the target energy action state, a calibration coefficient by determining values and value ranges of temperature and humidity, a ground water vapor pressure and the CO₂ flux, to calibrate the long-wave radiometer.

2. The calibration method for the long-wave radiometer as claimed in claim 1, wherein the collection process specifically comprises: obtaining the data of the meteorological parameters and the CO₂ flux measured at night at the target station.

3. The calibration method for the long-wave radiometer as claimed in claim 2, wherein the screening process further comprises: selecting data within 2 times standard deviation for the CO₂ flux; obtaining, based on regenerated data, a standard deviation after each cycle calculation; and
adopting a data processing method synchronized with the CO₂ flux for the meteorological parameters.

4. The calibration method for the long-wave radiometer as claimed in claim 3, wherein the calibration process specifically comprises: obtaining, based on the temperature and humidity, the ground water vapor pressure and the CO₂ flux, an absorption term and a CO₂ term for representing attenuation involved in the radiation transmission, and obtaining, based on a radiation energy balance principle, the calibration coefficient to calibrate the long-wave radiometer.

5. The calibration method for the long-wave radiometer as claimed in claim 4, where in a process of obtaining the absorption term, a formula of the absorption term e^{-kWm} is expressed as follows:
$${e}^{- k W m} = 1 - Δ {SI}_{0} cos ( Z ) ;$$
wherein I₀ represents a solar constant, Z represents a solar zenith angle, ΔS represents a solar radiation flux density of total absorption in an atmospheric column, and ΔS=0.172 (mW×0.1×30)^{0.303}, k represents a water vapor absorption coefficient, m represents an air mass, and W represents a water vapor content in the atmospheric column.

6. The calibration method for the long-wave radiometer as claimed in claim 5, where in a process of obtaining the CO₂ term, the CO₂ term is expressed as e^{-0.1bRetm};
wherein b represents an attenuation coefficient, and Re represents the CO₂ flux; and t represents a sampling time.

7. The calibration method for the long-wave radiometer as claimed in claim 6, wherein a process of obtaining the calibration coefficient comprises: obtaining, based on the radiation energy balance principle, the calibration coefficient by obtaining an energy equation between the CO₂ term and the absorption term in a horizontal plane above a canopy layer, wherein a formula of the energy equation is expressed as follows:
$${e}^{- 0 \text{.1} bRetm} \times cos ( Z ) = {C}_{1} {e}^{- k w m} \times cos ( Z ) + {C}_{0} ;$$
wherein C₁ and C₀ represent parameters related to various factors at the TOA.

8. The calibration method for the long-wave radiometer as claimed in claim 7, where in the process of obtaining the calibration coefficient, the calibration coefficient is expressed as follows:
$$\begin{matrix}L {R}_{0} / LR ; \\ LR = {C}_{1} + \vert {C}_{0} \vert ;\end{matrix}$$
wherein LR represents the long wave radiation value, and LR₀ represents long-wave radiation emitted by the canopy layer; and
the long-wave radiation emitted by the canopy layer is obtained based on a Boltzmann equation by measuring temperature and emissivity at the canopy layer.

9. The calibration method for the long-wave radiometer as claimed in claim 8, where in the process of obtaining the calibration coefficient, when C₁ and C₀ are positive and negative values, respectively, the calibration coefficient is determined based on the energy equation.

10. A calibration system for a long-wave radiometer, comprising:
a collection module, configured to obtain, by using a long-wave radiation value at the TOA as a standard, data of meteorological parameters and CO₂ flux measured by a target station;
a screening module, configured to determine, based on change rules of radiation, the meteorological parameters and the CO₂ flux, a target energy action state for expressing physical, chemical and biological processes involved in a radiation transmission and an interaction between the data and the radiation by obtaining an interrelationship among the radiation, the meteorological parameters and the CO₂ flux; and
a calibration module, configured to obtain, based on the target energy action state, a calibration coefficient by determining values and value ranges of temperature and humidity, a ground water vapor pressure and the CO₂ flux, to calibrate the long-wave radiometer.
